(19) United States
(12) Patent Application Publication
(10) Pub. No.: US 2025/0143667 A1
(43) Pub. Date: May 8, 2025

(54) COMPRESSION PLATE, MAMMOGRAPHY APPARATUS, AND IMAGE CAPTURING SYSTEM

(71) Applicant: FUJIFILM Corporation, Tokyo (JP)

(72) Inventors: Hisatsugu HORIUCHI, Kanagawa (JP); Lisako NOBUYAMA, Kanagawa (JP); Rika TASHIRO, Kanagawa (JP); Takeyasu KOBAYASHI, Kanagawa (JP); Sayaka SAITO, Kanagawa (JP)

(73) Assignee: FUJIFILM Corporation, Tokyo (JP)

(21) Appl. No.: 18/937,990

(22) Filed: Nov. 5, 2024

(30) Foreign Application Priority Data
Nov. 7, 2023 (JP) 2023-190076

(51) Int. Cl. A61B 8/08; A61B 8/00

(52) U.S. Cl. CPC A61B 8/0825; A61B 8/403; A61B 8/406; A61B 8/4444

(57) ABSTRACT

A compression plate includes a body part having a box shape with an open top, and a guide member that guides an ultrasound probe along a scanning direction of the ultrasound probe. The guide member includes a first guide part that guides the ultrasound probe along a first scanning path, and a second guide part that guides the ultrasound probe along a second scanning path. The first guide part and the second guide part are disposed such that effective image regions of the ultrasound probe partially overlap each other in a case in which scanning using the ultrasound probe is performed along the first scanning path and the second scanning path.

### CROSS-REFERENCE TO RELATED APPLICATION

This application is based on and claims priority under 35 USC 119 from Japanese Patent Application No. 2023-190076 filed on Nov. 7, 2023, the disclosure of which is incorporated by reference herein.

### BACKGROUND OF THE INVENTION

### 1. Field of the Invention

The present disclosure relates to a compression plate, a mammography apparatus, and an image capturing system.

### 2. Description of the Related Art

A mammography apparatus is known, which irradiates a breast of an examinee with radiation emitted from a radiation source and detects the radiation transmitted through the breast via a radiation detector to capture a radiation image.

In addition, an ultrasonography apparatus that captures an ultrasound image of a breast of an examinee by scanning the breast via an ultrasound probe.

The presence or absence of a tumor may be detected by comparing the radiation image and the ultrasound image of the breast. In this case, a shape of the breast is changed depending on the presence or absence of compression, and thus it is difficult to compare a position of the tumor between mammography and ultrasonography. Therefore, there is a method in which an ultrasound probe is mounted on a compression plate and an ultrasound examination is performed in a compressed state.

In a case in which the scanning using the ultrasound probe is performed via the compression plate, a user such as a doctor or a radiologic technologist performs the scanning using the ultrasound probe while viewing the ultrasound image displayed on a screen without looking at a hand gripping the ultrasound probe. For this reason, it may be unclear which part of the compression plate is scanned at what angle. On the other hand, for example, JP1996-098836A (JP-H8-098836A), JP2020-000446A, and JP2008-272459A describe guide members in a case of performing scanning using an ultrasound probe on a compression plate.

### SUMMARY OF THE INVENTION

All of the guide members described in JP1996-098836A (JP-H8-098836A), JP2020-000446A, and JP2008-272459A are for linearly guiding the ultrasound probe, and the scanning using the ultrasound probe cannot be performed such that the ultrasound images partially overlap each other. Therefore, scanning omission of the ultrasound probe may occur.

The present disclosure has been made in consideration of the above-described circumstances, and an object of the present disclosure is to provide a compression plate, a mammography apparatus, and an image capturing system capable of suppressing scanning omission of an ultrasound probe in a case of performing an ultrasound examination via the compression plate.

In order to achieve the above-described object, a first aspect of the present disclosure provides a compression plate that is used in a mammography apparatus, the compression plate comprising: a body part having a box shape with an open top; and a guide member that is provided at the body part and that guides an ultrasound probe along a scanning direction of the ultrasound probe, in which the guide member includes a first guide part that guides the ultrasound probe along a first scanning path, and a second guide part that guides the ultrasound probe along a second scanning path, and the first guide part and the second guide part are disposed such that effective image regions of the ultrasound probe partially overlap with each other in a case in which scanning using the ultrasound probe is performed along the first scanning path and the second scanning path.

A second aspect of the present disclosure provides the compression plate according to the first aspect, further comprising: a locking mechanism that attachably and detachably fixes the guide member to the body part.

A third aspect of the present disclosure provides the compression plate according to the first aspect, in which a space capable of accommodating a distal end part of the ultrasound probe is provided between the first guide part and a lower surface of the body part and between the second guide part and the lower surface of the body part, and the first guide part and the second guide part are provided at positions capable of being in contact with a depression provided at a side surface of the ultrasound probe.

A fourth aspect of the present disclosure provides the compression plate according to the first aspect, in which the guide member further includes a third guide part that guides the ultrasound probe along a third scanning path, and the second guide part and the third guide part are disposed such that effective image regions of the ultrasound probe partially overlap each other in a case in which the scanning using the ultrasound probe is performed along the second scanning path and the third scanning path.

A fifth aspect of the present disclosure provides the compression plate according to the fourth aspect, in which the guide member further includes a frame part, the second guide part extends from one end of the frame part toward another end of the frame part facing the one end, and the third guide part extends from the other end of the frame part toward the one end of the frame part.

A sixth aspect of the present disclosure provides the compression plate according to the fourth aspect, in which the guide member further includes a frame part, the second guide part extends from one end of the frame part toward another end of the frame part facing the one end, and the third guide part extends from the one end of the frame part toward the other end of the frame part.

A seventh aspect of the present disclosure provides the compression plate according to the fourth aspect, in which the guide member further includes a frame part, the second guide part connects one end of the frame part and another end of the frame part facing the one end, and the third guide part connects the one end of the frame part and the other end of the frame part.

An eighth aspect of the present disclosure provides the compression plate according to the first aspect, further comprising: an intermediate member that is interposed between the ultrasound probe and an examinee, in which the intermediate member is provided at an entire lower surface of the body part.

A ninth aspect of the present disclosure provides the compression plate according to the first aspect, in which the guide member further includes a position detection sensor that detects a position of the ultrasound probe.

In order to achieve the above-described object, a tenth aspect of the present disclosure provides a mammography apparatus comprising: the compression plate according to any one of the first to ninth aspects.

In order to achieve the above-described object, an eleventh aspect of the present disclosure provides an image capturing system comprising: an ultrasonography apparatus including an ultrasound probe; and a mammography apparatus including a compression plate, in which the compression plate includes a body part having a box shape with an open top, and a guide member that is provided at the body part and that guides the ultrasound probe along a scanning direction of the ultrasound probe, the guide member includes a first guide part that guides the ultrasound probe along a first scanning path, and a second guide part that guides the ultrasound probe along a second scanning path, and the first guide part and the second guide part are disposed such that effective image regions of the ultrasound probe partially overlap with each other in a case in which scanning using the ultrasound probe is performed along the first scanning path and the second scanning path.

A twelfth aspect of the present disclosure provides the image capturing system according to the eleventh aspect, in which the ultrasound probe includes a transceiver unit that transmits and receives ultrasound waves, and a gripping part that is continuous with the transceiver unit, and a width of the gripping part is smaller than a width of an effective image region of the transceiver unit.

A thirteenth aspect of the present disclosure provides the image capturing system according to the twelfth aspect, in which the ultrasound probe is provided with a depression at a side surface of the gripping part, and the depression is in contact with the first guide part and the second guide part in a case in which the scanning using the ultrasound probe is performed along the first scanning path and the second scanning path.

A fourteenth aspect of the present disclosure provides the image capturing system according to the thirteenth aspect, in which a radius of the depression is larger than a radius of a cross-sectional shape of each of the first guide part and the second guide part.

A fifteenth aspect of the present disclosure provides the image capturing system according to the eleventh aspect, in which the guide member further includes a third guide part that guides the ultrasound probe along the third scanning path, and in a case in which a width of the second guide part is denoted by W1, a distance between the second guide part and the third guide part is denoted by W2, a width of the third guide part is denoted by W3, and a width of the effective image regions of the ultrasound probe is denoted by W4, a relationship of W4 >W1/2+W2+W3/2 is satisfied.

A sixteenth aspect of the present disclosure provides the image capturing system according to the twelfth aspect, further comprising: an intermediate member that is interposed between the ultrasound probe and an examinee, in which the intermediate member is provided at the transceiver unit of the ultrasound probe.

According to the present disclosure, it is possible to suppress scanning omission of the ultrasound probe in a case of performing the ultrasound examination via the compression plate.

### BRIEF DESCRIPTION OF THE DRAWINGS

FIG. 1 is a view showing an example of an image capturing system according to a first embodiment.

FIG. 2 is a side view showing an example of a configuration of a compression unit.

FIG. 3 is a perspective view schematically showing an example of a compression plate according to the first embodiment.

FIG. 4 is a top view schematically showing an example of a guide member according to the first embodiment.

FIG. 5 is a perspective view schematically showing an example of a body part according to the embodiment.

FIG. 6 is a diagram showing an example of a positional relationship between an ultrasound probe, a second guide part, and a third guide part according to the embodiment.

FIG. 7 is a diagram schematically showing an example of an ultrasound image according to the embodiment.

FIG. 8 is a front view schematically showing an example of the ultrasound probe according to the embodiment.

FIG. 9 is a diagram showing a state in which an intermediate member fixed to the ultrasound probe is expanded.

FIG. 10 is a top view schematically showing an example of a guide member according to a second embodiment.

FIG. 11 is a top view schematically showing an example of a guide member according to a third embodiment.

FIG. 12 is a perspective view showing an example of a positional relationship between an ultrasound probe and a second guide part according to a fourth embodiment.

FIG. 13 is a front view showing an example of the positional relationship between the ultrasound probe and the second guide part according to the fourth embodiment.

### DESCRIPTION OF THE PREFERRED EMBODIMENTS

Hereinafter, embodiments of the present invention will be described in detail with reference to the accompanying drawings. It should be noted that each embodiment does not limit the invention.

### First Embodiment

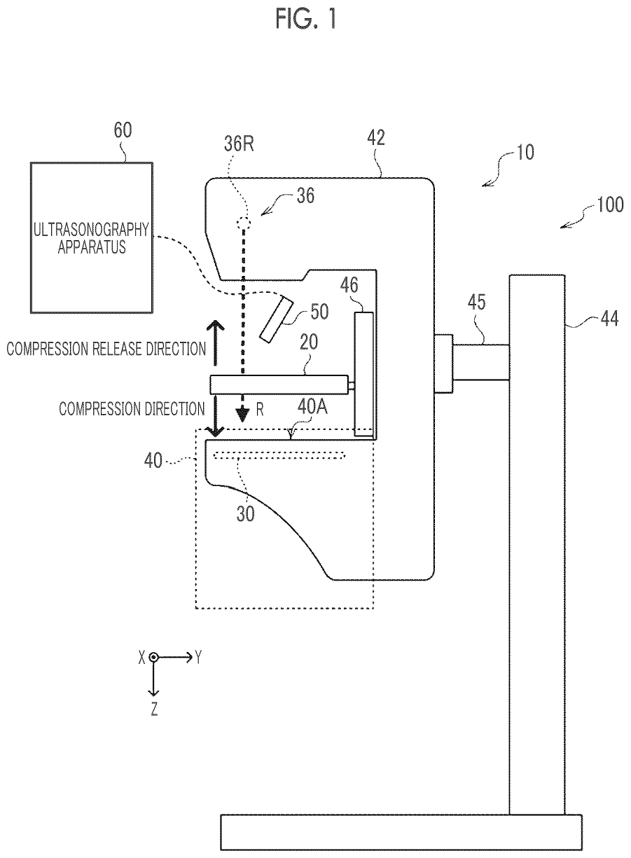

FIG. 1 is a view showing an example of an image capturing system 100 according to the first embodiment. The image capturing system 100 according to the present embodiment comprises a mammography apparatus 10 and an ultrasonography apparatus 60. It should be noted that, in FIG. 1, a depth direction is defined as an X axis direction, a left-right direction is defined as a Y axis direction, and an up-down direction is defined as a Z axis direction.

The mammography apparatus 10 according to the present embodiment is an apparatus that targets a breast of an examinee as a subject and captures a radiation image of the breast by irradiating the breast with radiation R (for example, X-rays). It should be noted that the mammography apparatus 10 may be an apparatus that images the breast of the examinee in a state (sitting state) in which the examinee is sitting on a chair (including a wheelchair) or the like in addition to a state (standing state) in which the examinee is standing.

As shown in FIG. 1, a mammography apparatus 10 according to the present embodiment comprises a compression plate 20, a radiation detector 30, a radiation emission unit 36, an imaging table 40, an arm part 42, a base 44, a shaft part 45, and a compression unit 46.

The radiation detector 30 detects the radiation R that has been transmitted through the breast of the examinee. The radiation detector 30 is disposed in the imaging table 40. In a case in which imaging is performed in the mammography apparatus 10 according to the present embodiment, the breast of the examinee is positioned on an imaging surface 40A of the imaging table 40 by the user such as a doctor or a radiologic technologist. For example, the imaging surface 40A or the like that is in contact with the breast of the examinee are made of carbon or the like in terms of the transmittance or the intensity of the radiation R.

The radiation detector 30 detects the radiation R transmitted through the breast of the examinee and the imaging table 40, generates the radiation image based on the detected radiation R, and outputs image data representing the generated radiation image. A type of the radiation detector 30 is not particularly limited and may be, for example, an indirect conversion type radiation detector that converts the radiation R into light and converts the converted light into a charge, or a direct conversion type radiation detector that directly converts the radiation R into a charge.

The radiation emission unit 36 comprises a radiation source 36R. The radiation emission unit 36 is provided on the arm part 42 together with the imaging table 40 and the compression unit 46. It should be noted that the mammography apparatus 10 comprises the arm part 42, the base 44, and the shaft part 45. The arm part 42 is held by the base 44 to be movable in the up-down direction (Z axis direction). The arm part 42 is connected to the base 44 by the shaft part 45. The compression unit 46 and the arm part 42 can be rotated relative to the base 44, separately, using the shaft part 45 as a rotation axis. In the present embodiment, gears (not shown) are provided in each of the shaft part 45, the arm part 42, and the compression unit 46, and the gears are switched between an engaged state and a disengaged state to connect each of the arm part 42 and the compression unit 46 to the shaft part 45. One or two of the arm part 42 or the compression unit 46 connected to the shaft part 45 are rotated integrally with the shaft part 45.

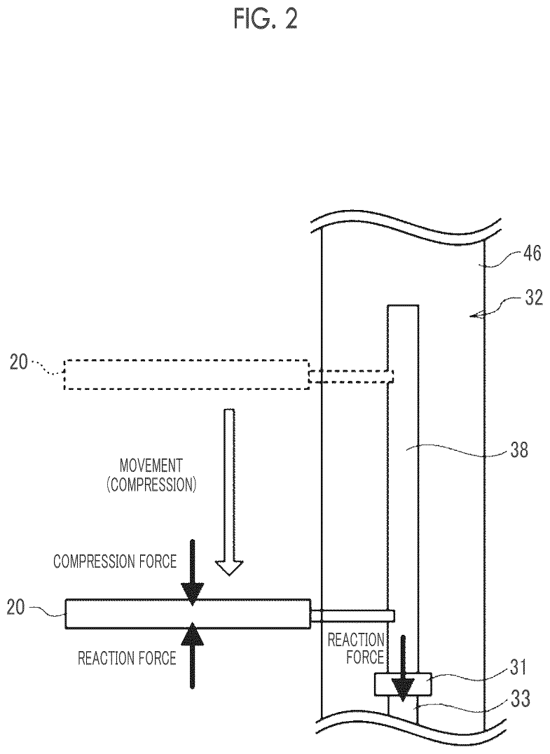

FIG. 2 is a side view showing an example of a configuration of the compression unit 46. It should be noted that FIG. 2 is a side view of the compression unit 46 as seen in the X axis direction of FIG. 1.

As shown in FIG. 2, the compression unit 46 is provided with a compression plate driving unit 32, a compression force detection sensor 33, and the compression plate 20. The compression unit 46 comprises the compression plate driving unit 32, including a motor 31 and a ball screw 38, and the compression force detection sensor 33. The compression force detection sensor 33 has a function of detecting a reaction force to a compression force that compresses the entire breast by the compression plate 20, to detect the compression force.

The compression plate 20 according to the present embodiment is a compression member, and is moved in the up-down direction (Z axis direction) by the compression plate driving unit 32 to compress the breast of the examinee between the compression plate 20 and the imaging table 40. As shown in FIG. 1, in the movement direction of the compression plate 20, a direction of compressing the breast, in other words, a direction of approaching the imaging surface 40A is referred to as a “compression direction”, and a direction of releasing the compression of the breast, in other words, a direction of approaching the radiation emission unit 36 is referred to as a “compression release direction”.

It is preferable that the compression plate 20 is optically transparent in order to check positioning or a compressed state in the compression of the breast, and the compression plate 20 is made of a material having high transmittance for the radiation R. Further, it is desirable that the compression plate 20 is made of a material that facilitates the transmission of the ultrasound from an ultrasound probe 50 of the ultrasonography apparatus 60. Examples of the material forming the compression plate 20 include resins such as polymethylpentene, polycarbonate, acrylic, and polyethylene terephthalate. In particular, polymethylpentene is suitable as the material forming the compression plate 20 since polymethylpentene has low rigidity, high elasticity, and high flexibility and has suitable values for acoustic impedance that affects the reflectance of the ultrasound and an attenuation coefficient that affects the attenuation of the ultrasound.

It should be noted that the compression plate 20 is not limited to the compression plate that compresses the entire breast, but may be the compression plate that compresses a part of the breast. In other words, the compression plate 20 may be smaller than the breast. As such a compression plate 20, for example, a compression plate 20 used for so-called spot imaging is known in which a radiation image of only a region in which a lesion exists is captured.

On the other hand, the ultrasonography apparatus 60 is an apparatus that includes the ultrasound probe 50 and targets the breast of the examinee as the subject to capture an ultrasound image by the user.

The ultrasound probe 50 is moved on the compression plate 20 by the user to acquire the ultrasound image of the breast by scanning the breast with the ultrasound.

The ultrasound probe 50 comprises a plurality of ultrasound transducers arranged one-dimensionally or two-dimensionally. Each of the ultrasound transducers transmits the ultrasound based on an applied driving signal, receives an ultrasound echo, and outputs a reception signal.

Each of the plurality of ultrasound transducers includes, for example, a transducer in which electrodes are formed at both ends of a piezoelectric material (piezoelectric body) such as a piezoelectric ceramic represented by lead (Pb) zirconate titanate (PZT) or a polymer piezoelectric element represented by polyvinylidene difluoride (PVDF). In a case in which the pulsed or continuous wave driving signal is transmitted to apply a voltage to the electrodes of the transducer, the piezoelectric body is expanded and contracted. The pulsed or continuous wave ultrasound is generated from each transducer by the expansion and contraction and these types of the ultrasound are synthesized to form an ultrasound beam. Each transducer receives the propagating ultrasound waves and is then expanded and contracted to generate an electric signal. The electric signal is output as an ultrasound reception signal and then input to a body of the ultrasonography apparatus 60 through a cable indicated by a dotted line.

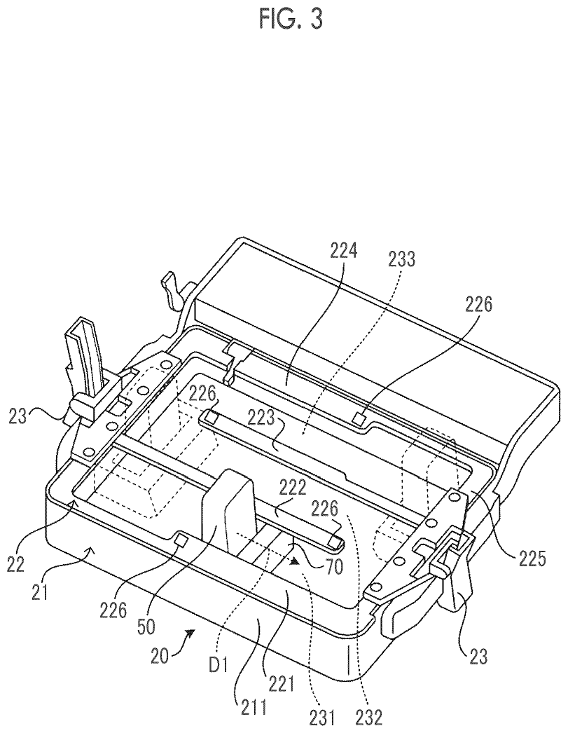

FIG. 3 is a perspective view schematically showing an example of the compression plate 20 according to the first embodiment.

As shown in FIG. 3, the compression plate 20 according to the present embodiment comprises a body part 21, a guide member 22, and a locking mechanism 23. The body part 21 is a box-shaped member with an open top and has a chest wall surface 211. The chest wall surface 211 is a surface facing a chest wall of the examinee. The guide member 22 is provided on the body part 21 and guides the ultrasound probe 50 along a scanning direction D1 of the ultrasound probe 50.

The locking mechanism 23 attachably and detachably fixes the guide member 22 to the body part 21. The locking mechanism 23 is provided on the guide member 22 as an example, but may be provided on the body part 21. The mammography can be performed in a state in which the guide member 22 is removed from the body part 21 in a case of the mammography, and the ultrasonography can be performed in a state in which the guide member 22 is attached to the body part 21 in a case of the ultrasonography. In addition, by comprising the locking mechanism 23, it is possible to prevent the guide member 22 from falling off from the body part 21 even in a case in which the compression plate 20 is inclined.

The guide member 22 includes a first guide part 221, a second guide part 222, a third guide part 223, a fourth guide part 224, a frame part 225, and a position detection sensor 226. The first guide part 221, the second guide part 222, the third guide part 223, and the fourth guide part 224 are provided in a direction along the chest wall surface 211 of the body part 21 as an example, but may be provided in a direction intersecting the chest wall surface 211 of the body part 21. A cross-sectional shape of each of the first guide part 221, the second guide part 222, the third guide part 223, and the fourth guide part 224 is not particularly limited, but need only be, for example, a circular shape, an elliptical shape, a triangular shape, or a rectangular shape.

The frame part 225 is a frame-like part provided along the periphery of the body part 21. The first guide part 221 is a part that protrudes in a direction of the second guide part 222 from the frame part 225. The second guide part 222 is a part that extends from one end of the frame part 225 toward the other end of the frame part 225 facing the one end. The third guide part 223 is a part that extends from the other end of the frame part 225 toward one end of the frame part 225 facing the other end. The fourth guide part 224 is a part that protrudes in a direction of the third guide part 223 from the frame part 225.

The first guide part 221 and the second guide part 222 guide the ultrasound probe 50 along a first scanning path 231. The second guide part 222 and the third guide part 223 guide the ultrasound probe 50 along a second scanning path 232. The third guide part 223 and the fourth guide part 224 guide the ultrasound probe 50 along a third scanning path 233.

The first guide part 221 and the second guide part 222 are disposed such that effective image regions (described later) of the ultrasound probe 50 partially overlap each other in a case in which scanning using the ultrasound probe 50 is performed along the first scanning path 231 and the second scanning path 232. Similarly, the second guide part 222 and the third guide part 223 are disposed such that effective image regions of the ultrasound probe 50 partially overlap each other in a case in which scanning using the ultrasound probe 50 is performed along the second scanning path 232 and the third scanning path 233. Since the effective image regions of the ultrasound probe 50 can partially overlap each other, scanning omission of the ultrasound probe 50 can be suppressed.

Each of the first guide part 221, the second guide part 222, the third guide part 223, and the fourth guide part 224 is provided with the position detection sensor 226. The position detection sensor 226 is a sensor for detecting a position of the ultrasound probe 50, and various sensors such as an optical sensor, an ultrasound sensor, and a magnetic sensor are used as the position detection sensor 226. The position detection sensor 226 detects whether the ultrasound probe 50 is located on the first scanning path 231, the second scanning path 232, or the third scanning path 233. A detection result of the position detection sensor 226 is displayed on, for example, a display unit (not shown) of the ultrasonography apparatus 60, and the user can understand the position of the ultrasound probe 50 while viewing the ultrasound image without looking at a hand gripping the ultrasound probe 50. However, the number and the attachment locations of the position detection sensors 226 are not limited to the example of FIG. 3, and the position detection sensors 226 need only be at a location at which the position of the ultrasound probe 50 can be appropriately detected.

It should be noted that, in the example of FIG. 3, a distal end part of the ultrasound probe 50 is held by a holding member 70, and the scanning using the ultrasound probe 50 is performed in a state in which the distal end part is held by the holding member 70. The holding member 70 is a member that holds the distal end part of the ultrasound probe 50 such that the holding member 70 surrounds the periphery of the distal end part of the ultrasound probe 50, and a posture of the ultrasound probe 50 can be stabilized by providing the holding member 70.

In the example of FIG. 3, the first guide part 221 and the fourth guide part 224 are provided on the guide member 22 side, but the first guide part 221 and the fourth guide part 224 may be provided on the body part 21 side. In this case, the second guide part 222 is replaced with the first guide part 221, and the third guide part 223 is replaced with the second guide part 222.

In addition, in the example of FIG. 3, the four guide parts, that is, the first guide part 221, the second guide part 222, the third guide part 223, and the fourth guide part 224 are provided, but the number of the guide parts is not limited to four. The number of the guide parts need only be two or more, and may be five or more depending on the shape of the ultrasound probe 50, the size of the compression plate 20, and the like.

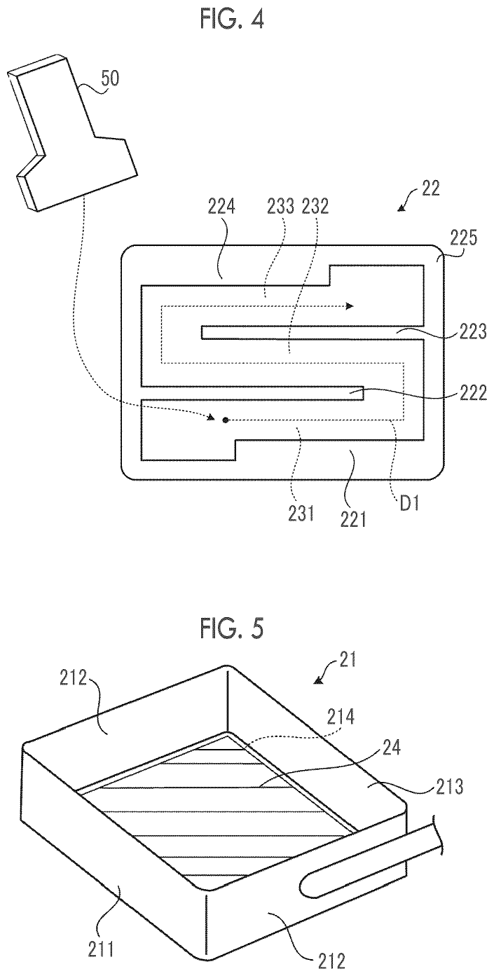

FIG. 4 is a top view schematically showing an example of the guide member 22 according to the first embodiment. It should be noted that, in FIG. 4, for simplicity of illustration, only the guide member 22 from which the body part 21 is separated is shown.

As described above, the guide member 22 shown in FIG. 4 includes the first guide part 221, the second guide part 222, the third guide part 223, the fourth guide part 224, and the frame part 225. The second guide part 222 extends from one end of the frame part 225 toward the other end of the frame part 225 facing the one end, and the third guide part 223 extends from the other end of the frame part 225 toward one end of the frame part 225 facing the other end. The second guide part 222 is connected to one end of the frame part 225 and is not connected to the other end of the frame part 225 facing the one end. On the other hand, the third guide part 223 is connected to the other end of the frame part 225, and is not connected to the one end of the frame part 225 facing the other end. That is, the second guide part 222 and the third guide part 223 are disposed in a staggered manner. The second guide part 222 and the third guide part 223 are disposed in a staggered manner, so that the scanning using the ultrasound probe 50 can be performed in a zigzag manner. Here, the term “zigzag” refers to a state in which a straight line is bent in a Z shape.

FIG. 5 is a perspective view schematically showing an example of the body part 21 according to the present embodiment. It should be noted that, in FIG. 5, for simplicity of illustration, only the body part 21 separated from the guide member 22 is shown.

The body part 21 shown in FIG. 5 includes a chest wall surface 211, two side surfaces 212, a rear surface 213, and a lower surface 214. The chest wall surface 211, the two side surfaces 212, and the rear surface 213 are disposed to surround the lower surface 214. The compression plate 20 comprises an intermediate member 24 that is interposed between the ultrasound probe 50 and the examinee. The intermediate member 24 is, for example, provided on the entire lower surface 214 of the body part 21.

As the intermediate member 24, for example, a gel sheet is used in which a gel-like (or jelly-like) acoustic matching material is accommodated. Here, the acoustic matching material is a member that fills a space between the lower surface 214 and the ultrasound probe 50, fills a space between the examinee and the lower surface 214, and is acoustically coupled to the examinee. It is preferable that the acoustic matching material is a material having the acoustic impedance close to the acoustic impedance of the examinee, the lower surface 214, and the ultrasound probe 50. By providing the intermediate member 24, a degree of contact between the breast of the examinee and the compression plate 20 can be improved. On the other hand, by providing the intermediate member 24, a degree of contact between the compression plate 20 and the ultrasound probe 50 can be improved.

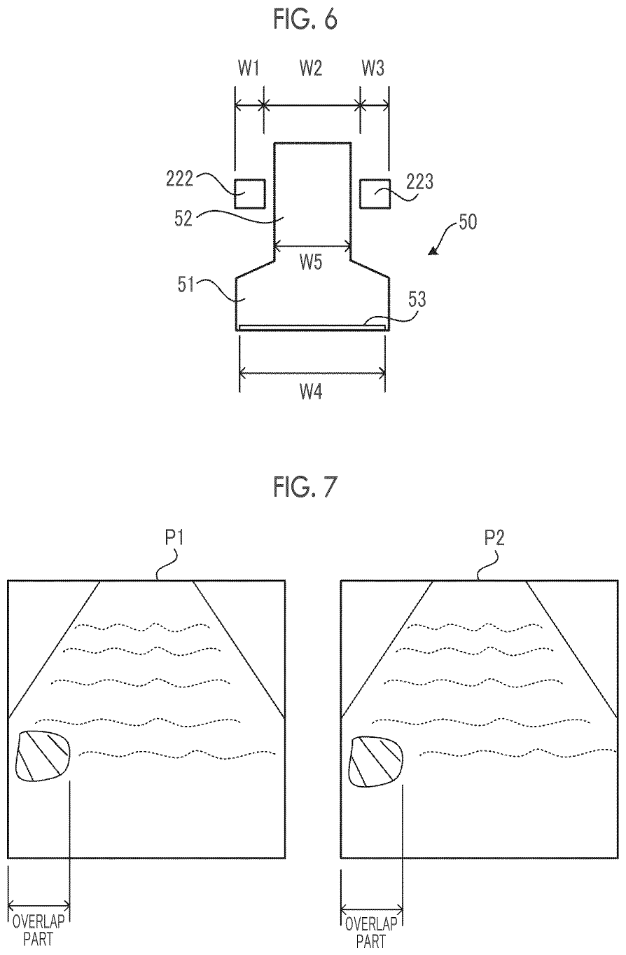

FIG. 6 is a diagram showing an example of a positional relationship between the ultrasound probe 50, the second guide part 222, and the third guide part 223 according to the present embodiment.

The ultrasound probe 50 shown in FIG. 6 includes a transceiver unit 51 and a gripping part 52. The transceiver unit 51 is a unit that transmits and receives the ultrasound waves. The gripping part 52 is a part that is continuous with the transceiver unit 51 and is gripped by the user. A width W5 of the gripping part 52 is smaller than a width W4 of an effective image region 53 of the transceiver unit 51.

Here, in a case in which a width of the second guide part 222 is denoted by W1, a distance between the second guide part 222 and the third guide part 223 is denoted by W2, a width of the third guide part 223 is denoted by W3, and a width of the effective image region 53 of the ultrasound probe 50 is denoted by W4, the following relationship represented by Expression (1) is satisfied.

W4>W1/2+W2+W3/2 (1)

By disposing the second guide part 222 and the third guide part 223 such that Expression (1) is satisfied, the effective image regions 53 of the ultrasound probe 50 can partially overlap each other in a case in which the scanning using the ultrasound probe 50 is performed along the first scanning path 231 and the second scanning path 232.

FIG. 7 is a diagram schematically showing an example of ultrasound images P1 and P2 according to the present embodiment.

As described above, by partially overlapping the effective image regions 53 of the ultrasound probe 50, as shown in FIG. 7, two ultrasound images P1 and P2 that partially overlap each other are obtained. The ultrasound image P1 is an image obtained in a case in which the scanning using the ultrasound probe 50 is performed along the first scanning path 231. On the other hand, the ultrasound image P2 is an image obtained in a case in which the scanning using the ultrasound probe 50 is performed along the second scanning path 232.

As described above, since the two ultrasound images P1 and P2 partially overlap each other, scanning omission of the ultrasound probe 50 is suppressed.

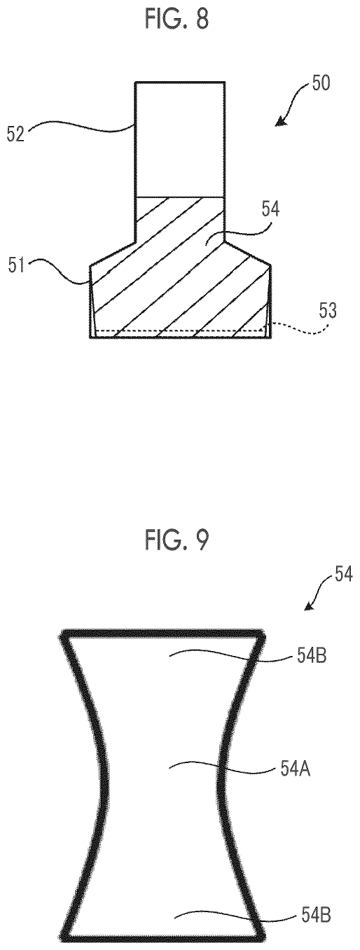

FIG. 8 is a front view schematically showing an example of the ultrasound probe 50 according to the present embodiment.

As shown in FIG. 8, the transceiver unit 51 of the ultrasound probe 50 is provided with an intermediate member 54. As the intermediate member 54, as described above, for example, a gel sheet is used in which a gel-like (or jelly-like) acoustic matching material is accommodated. It should be noted that, in a case in which the intermediate member 54 is provided in the ultrasound probe 50, the intermediate member 24 need not be provided on the lower surface 214 of the body part 21.

FIG. 9 is a diagram showing a state in which the intermediate member 54 fixed to the ultrasound probe 50 is expanded.

In a case in which the ultrasound probe 50 has a narrowed part (corresponding to a part of W5 in FIG. 6), it is desirable that the intermediate member 54 is firmly fixed to the distal end of the ultrasound probe 50 and is kept in a state of not moving even in a case in which the scanning using the ultrasound probe 50 is performed. In a case in which the ultrasound probe 50 has the narrowed part (corresponding to a part of W5 in FIG. 6), as shown in FIG. 9, the shape of the intermediate member 54 is not a simple rectangular shape, and is suitably a shape in which a width of a center part 54A is the shortest (narrowest) and the width is gradually increased (widened) toward both end parts 54B, in other words, a shape in which the center part 54A is narrowed with respect to both end parts 54B. The center part 54A is disposed to correspond to the effective image region 53 of the ultrasound probe 50. Both end parts 54B are wound around and fixed to a lower-side part of the gripping part 52 of the ultrasound probe 50 (that is, the narrowed part of the ultrasound probe 50) near the lower-side part. By forming the intermediate member 54 in this shape, the intermediate member 54 can be firmly wound around the ultrasound probe 50.

It should be noted that the intermediate member 54 may be provided to include the transceiver unit 51 and the holding member 70 in a state in which the transceiver unit 51 of the ultrasound probe 50 is held by the holding member 70 (see FIG. 3).

As described above, according to the present embodiment, the first guide part 221 to the fourth guide part 224 are disposed such that the effective image regions 53 of the ultrasound probe 50 partially overlap each other in a case in which the scanning using the ultrasound probe 50 is performed along the first scanning path 231, the second scanning path 232, and the third scanning path 233. Therefore, scanning omission of the ultrasound probe 50 is suppressed. In addition, according to the present embodiment, the second guide part 222 and the third guide part 223 of the guide member 22 are disposed in a staggered manner. Therefore, the scanning using the ultrasound probe 50 is performed in a zigzag manner, and the ultrasound examination can be efficiently performed.

### Second Embodiment

In the second embodiment, an embodiment will be described in which the second guide part and the third guide part are disposed in a comb shape.

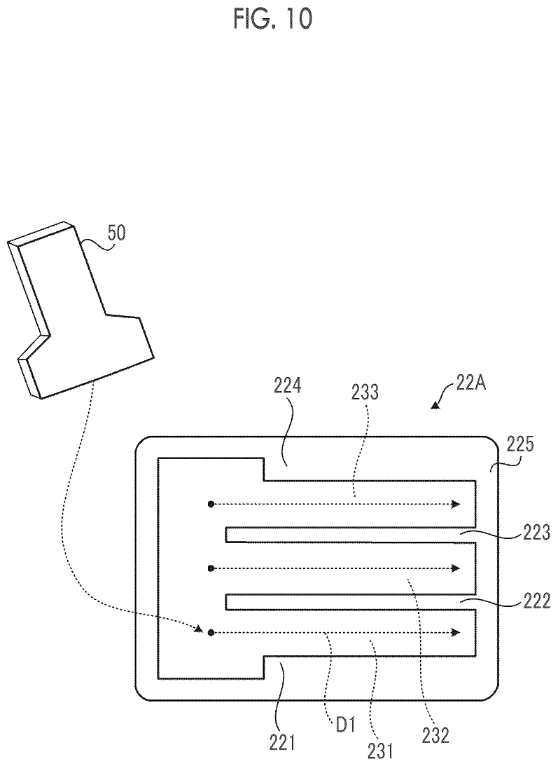

FIG. 10 is a top view schematically showing an example of a guide member 22A according to the second embodiment. It should be noted that, in FIG. 10, for simplicity of illustration, only the guide member 22A from which the body part 21 is separated is shown.

As described above, the guide member 22A shown in FIG. 10 includes the first guide part 221, the second guide part 222, the third guide part 223, the fourth guide part 224, and the frame part 225. The second guide part 222 extends from one end of the frame part 225 toward the other end of the frame part 225 facing the one end, and the third guide part 223 extends from one end of the frame part 225 toward the other end of the frame part 225 facing the one end. Both the second guide part 222 and the third guide part 223 are connected to the one end of the frame part 225, and are not connected to the other end of the frame part 225 facing the one end. That is, the second guide part 222 and the third guide part 223 are disposed in a comb shape. By disposing the second guide part 222 and the third guide part 223 in a comb shape, the scanning using the ultrasound probe 50 can always be performed in the same direction.

As described above, according to the present embodiment, as in the first embodiment, the first guide part 221 to the fourth guide part 224 are disposed such that the effective image regions 53 of the ultrasound probe 50 partially overlap each other in a case in which the scanning using the ultrasound probe 50 is performed along the first scanning path 231, the second scanning path 232, and the third scanning path 233. Therefore, scanning omission of the ultrasound probe 50 is suppressed. According to the present embodiment, the second guide part 222 and the third guide part 223 of the guide member 22A are disposed in a comb shape. Therefore, the scanning using the ultrasound probe 50 is always performed in the same direction, and the ultrasound examination can be efficiently performed.

### Third Embodiment

In the third embodiment, an embodiment will be described in which the second guide part and the third guide part are disposed in a ladder shape.

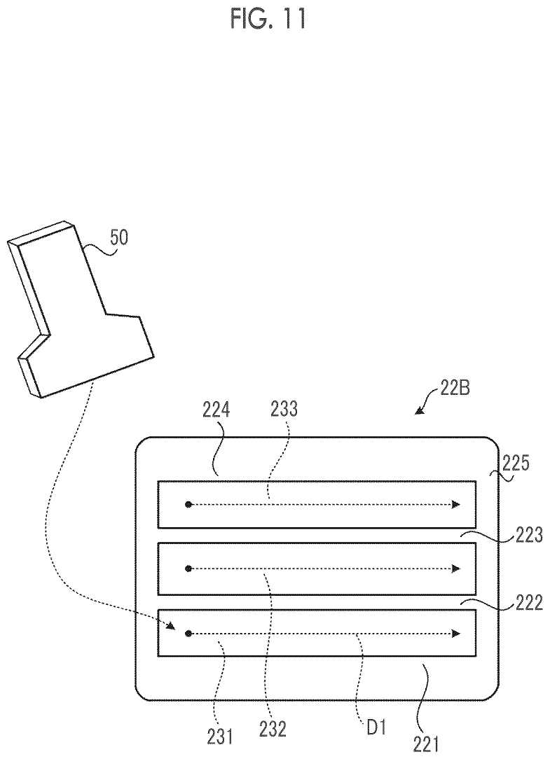

FIG. 11 is a top view schematically showing an example of a guide member 22B according to the third embodiment. It should be noted that, in FIG. 11, for simplicity of illustration, only the guide member 22B from which the body part 21 is separated is shown.

As described above, the guide member 22B shown in FIG. 11 includes the first guide part 221, the second guide part 222, the third guide part 223, the fourth guide part 224, and the frame part 225. The second guide part 222 connects one end of the frame part 225 to the other end of the frame part 225 facing the one end, and the third guide part 223 connects one end of the frame part 225 to the other end of the frame part 225 facing the one end. That is, the second guide part 222 and the third guide part 223 are disposed in a ladder shape. By disposing the second guide part 222 and the third guide part 223 in a ladder shape, the strength of the guide member 22B can be improved as compared with a case in which the second guide part 222 and the third guide part 223 are disposed in a comb shape.

As described above, according to the present embodiment, as in the first embodiment, the first guide part 221 to the fourth guide part 224 are disposed such that the effective image regions 53 of the ultrasound probe 50 partially overlap each other in a case in which the scanning using the ultrasound probe 50 is performed along the first scanning path 231, the second scanning path 232, and the third scanning path 233. Therefore, scanning omission of the ultrasound probe 50 is suppressed. In addition, according to the present embodiment, the second guide part 222 and the third guide part 223 of the guide member 22B are disposed in a ladder shape. Therefore, it is possible to improve the strength of the guide member 22B.

### Fourth Embodiment

In the fourth embodiment, an embodiment will be described in which the ultrasound probe is supported so as not to fall off.

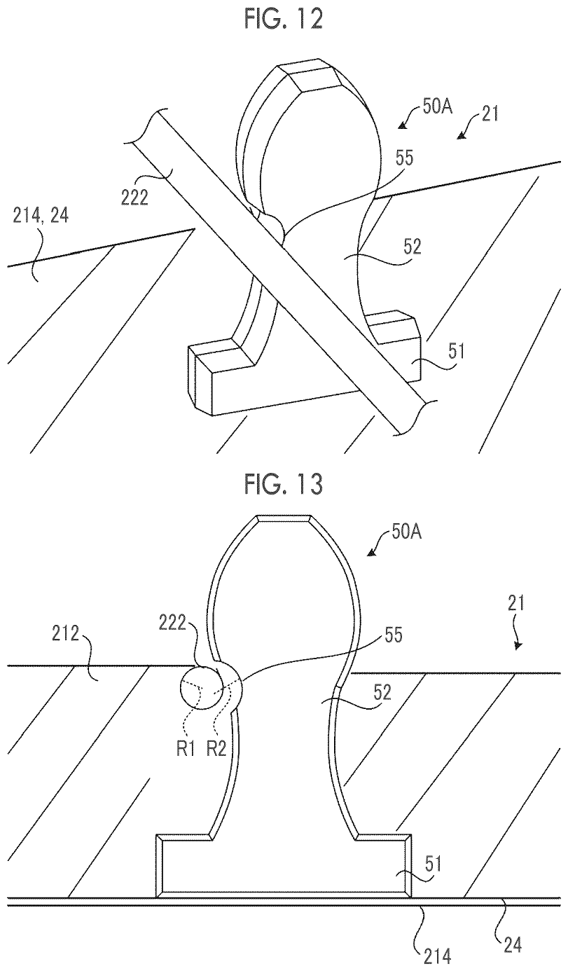

FIG. 12 is a perspective view showing an example of a positional relationship between an ultrasound probe 50A and the second guide part 222 according to the fourth embodiment. In addition, FIG. 13 is a front view showing an example of the positional relationship between the ultrasound probe 50A and the second guide part 222 according to the fourth embodiment.

As shown in FIGS. 12 and 13, a space capable of accommodating the distal end part of the ultrasound probe 50A is provided between the second guide part 222 and the lower surface 214 of the body part 21. It should be noted that, although not shown here, a space capable of accommodating the distal end part of the ultrasound probe 50A is also provided between each of the first guide part 221, the third guide part 223, and the fourth guide part 224, and the lower surface 214 of the body part 21.

In the ultrasound probe 50A according to the present embodiment, a depression 55 is provided on a side surface of the gripping part 52. The second guide part 222 is provided at a position at which the second guide part 222 can be in contact with the depression 55 provided on the side surface of the ultrasound probe 50A. It should be noted that, although not shown here, the first guide part 221, the third guide part 223, and the fourth guide part 224 are also provided at positions at which the first guide part 221, the third guide part 223, and the fourth guide part 224 can be in contact with the depression 55 provided on the side surface of the ultrasound probe 50A. For example, in a case in which the scanning using the ultrasound probe 50A is performed along the first scanning path 231 and the second scanning path 232, the depression 55 is in contact with the first guide part 221 and the second guide part 222. The depression 55 is in contact with the first guide part 221 and the second guide part 222, so that the ultrasound probe 50A is supported so as not to fall off.

Here, it is desirable that a shape of the depression 55 is, for example, a circular shape. Further, it is desirable that the cross-sectional shape of the second guide part 222 is, for example, a circular shape. In this case, it is desirable that a radius R2 of the depression 55 is larger than a radius R1 of the cross-sectional shape of the second guide part 222. The same applies to the first guide part 221, the third guide part 223, and the fourth guide part 224.

In a case in which the radius R2 of the depression 55 and the radius RI of the second guide part 222 are substantially the same as each other, a height of the ultrasound probe 50A is defined at the position of the depression 55. In a case in which the height of the ultrasound probe 50A is defined, the degree of contact with the compression plate 20 is lowered, which is not preferable. Therefore, by setting the radius R2 of the depression 55 to be larger than the radius R1 of the cross-sectional shape of the second guide part 222, it is possible to ensure the degree of freedom of the ultrasound probe 50A in a height direction.

As described above, according to the present embodiment, the ultrasound probe 50A is supported while ensuring the degree of freedom of the ultrasound probe 50A in the height direction, and the decrease in the degree of contact with the compression plate 20 is suppressed.

In addition, the configurations and operations of the compression plate 20, the mammography apparatus 10, and the image capturing system 100 described in each of the above-described embodiments are merely examples, and it goes without saying that the changes can be made depending on a situation within a range that does not depart from the gist of the present invention.

In regard to the embodiments described above, the following supplementary notes will be further disclosed.

### Supplementary Note 1

A compression plate that is used in a mammography apparatus, the compression plate comprising: a body part having a box shape with an open top; and a guide member that is provided at the body part and that guides an ultrasound probe along a scanning direction of the ultrasound probe, in which the guide member includes a first guide part that guides the ultrasound probe along a first scanning path, and a second guide part that guides the ultrasound probe along a second scanning path, and the first guide part and the second guide part are disposed such that effective image regions of the ultrasound probe partially overlap with each other in a case in which scanning using the ultrasound probe is performed along the first scanning path and the second scanning path.

### Supplementary Note 2

The compression plate according to supplementary note 1, further comprising: a locking mechanism that attachably and detachably fixes the guide member to the body part.

### Supplementary Note 3

The compression plate according to supplementary note 1 or 2, in which a space capable of accommodating a distal end part of the ultrasound probe is provided between the first guide part and a lower surface of the body part and between the second guide part and the lower surface of the body part, and the first guide part and the second guide part are provided at positions capable of being in contact with a depression provided at a side surface of the ultrasound probe.

### Supplementary Note 4

The compression plate according to any one of supplementary notes 1 to 3, in which the guide member further includes a third guide part that guides the ultrasound probe along a third scanning path, and the second guide part and the third guide part are disposed such that effective image regions of the ultrasound probe partially overlap each other in a case in which the scanning using the ultrasound probe is performed along the second scanning path and the third scanning path.

### Supplementary Note 5

The compression plate according to supplementary note 4, in which the guide member further includes a frame part, the second guide part extends from one end of the frame part toward the other end of the frame part facing the one end, and the third guide part extends from another end of the frame part toward the one end of the frame part.

### Supplementary Note 6

The compression plate according to supplementary note 4, in which the guide member further includes a frame part, the second guide part extends from one end of the frame part toward another end of the frame part facing the one end, and the third guide part extends from the one end of the frame part toward the other end of the frame part.

### Supplementary Note 7

The compression plate according to supplementary note 4, in which the guide member further includes a frame part, the second guide part connects one end of the frame part and another end of the frame part facing the one end, and the third guide part connects the one end of the frame part and the other end of the frame part.

### Supplementary Note 8

The compression plate according to any one of supplementary notes 1 to 7, further comprising: an intermediate member that is interposed between the ultrasound probe and an examinee, in which the intermediate member is provided at an entire lower surface of the body part.

### Supplementary Note 9

The compression plate according to any one of supplementary notes 1 to 8, in which the guide member further includes a position detection sensor that detects a position of the ultrasound probe.

### Supplementary Note 10

A mammography apparatus comprising: the compression plate according to any one of supplementary notes 1 to 9.

### Supplementary Note 11

An image capturing system comprising: an ultrasonography apparatus including an ultrasound probe; and a mammography apparatus including a compression plate, in which the compression plate includes a body part having a box shape with an open top, and a guide member that is provided at the body part and that guides the ultrasound probe along a scanning direction of the ultrasound probe, the guide member includes a first guide part that guides the ultrasound probe along a first scanning path, and a second guide part that guides the ultrasound probe along a second scanning path, and the first guide part and the second guide part are disposed such that effective image regions of the ultrasound probe partially overlap with each other in a case in which scanning using the ultrasound probe is performed along the first scanning path and the second scanning path.

### Supplementary Note 12

The image capturing system according to supplementary note 11, in which the ultrasound probe includes a transceiver unit that transmits and receives ultrasound waves, and a gripping part that is continuous with the transceiver unit, and a width of the gripping part is smaller than a width of an effective image region of the transceiver unit.

### Supplementary Note 13

The image capturing system according to supplementary note 12, in which the ultrasound probe is provided with a depression at a side surface of the gripping part, and the depression is in contact with the first guide part and the second guide part in a case in which the scanning using the ultrasound probe is performed along the first scanning path and the second scanning path.

### Supplementary Note 14

The image capturing system according to supplementary note 13, in which a radius of the depression is larger than a radius of a cross-sectional shape of each of the first guide part and the second guide part.

### Supplementary Note 15

The image capturing system according to any one of supplementary notes 11 to 14, in which the guide member further includes a third guide part that guides the ultrasound probe along the third scanning path, and in a case in which a width of the second guide part is denoted by W1, a distance between the second guide part and the third guide part is denoted by W2, a width of the third guide part is denoted by W3, and a width of the effective image regions of the ultrasound probe is denoted by W4, a relationship of W4>W1/2+W2+W3/2 is satisfied.

### Supplementary Note 16

The image capturing system according to any one of supplementary notes 12 to 14, further comprising: an intermediate member that is interposed between the ultrasound probe and an examinee, in which the intermediate member is provided at the transceiver unit of the ultrasound probe.

What is claimed is:

1. A compression plate that is used in a mammography apparatus, the compression plate comprising:
a body part having a box shape with an open top; and
a guide member that is provided at the body part and that guides an ultrasound probe along a scanning direction of the ultrasound probe,
wherein the guide member includes:
a first guide part that guides the ultrasound probe along a first scanning path, and
a second guide part that guides the ultrasound probe along a second scanning path, and
wherein the first guide part and the second guide part are disposed such that effective image regions of the ultrasound probe partially overlap with each other in a case in which scanning using the ultrasound probe is performed along the first scanning path and the second scanning path.

2. The compression plate according to claim 1, further comprising:
a locking mechanism that attachably and detachably fixes the guide member to the body part.

3. The compression plate according to claim 1, wherein:
a space capable of accommodating a distal end part of the ultrasound probe is provided between the first guide part and a lower surface of the body part and between the second guide part and the lower surface of the body part, and
the first guide part and the second guide part are provided at positions capable of being in contact with a depression provided at a side surface of the ultrasound probe.

4. The compression plate according to claim 1, wherein:
the guide member further includes a third guide part that guides the ultrasound probe along a third scanning path, and
the second guide part and the third guide part are disposed such that effective image regions of the ultrasound probe partially overlap each other in a case in which the scanning using the ultrasound probe is performed along the second scanning path and the third scanning path.

5. The compression plate according to claim 4, wherein:
the guide member further includes a frame part,
the second guide part extends from one end of the frame part toward another end of the frame part facing the one end, and
the third guide part extends from the other end of the frame part toward the one end of the frame part.

6. The compression plate according to claim 4, wherein:
the guide member further includes a frame part,
the second guide part extends from one end of the frame part toward another end of the frame part facing the one end, and
the third guide part extends from the one end of the frame part toward the other end of the frame part.

7. The compression plate according to claim 4, wherein:
the guide member further includes a frame part,
the second guide part connects one end of the frame part and another end of the frame part facing the one end, and
the third guide part connects the one end of the frame part and the other end of the frame part.

8. The compression plate according to claim 1, further comprising:
an intermediate member that is interposed between the ultrasound probe and an examinee,
wherein the intermediate member is provided at an entire lower surface of the body part.

9. The compression plate according to claim 1,
wherein the guide member further includes a position detection sensor that detects a position of the ultrasound probe.

10. A mammography apparatus comprising:
the compression plate according to claim 1.

11. An image capturing system comprising:
an ultrasonography apparatus including an ultrasound probe; and
a mammography apparatus including a compression plate,
wherein the compression plate includes:
a body part having a box shape with an open top, and
a guide member that is provided at the body part and that guides the ultrasound probe along a scanning direction of the ultrasound probe,
wherein the guide member includes:
a first guide part that guides the ultrasound probe along a first scanning path, and
a second guide part that guides the ultrasound probe along a second scanning path, and
wherein the first guide part and the second guide part are disposed such that effective image regions of the ultrasound probe partially overlap with each other in a case in which scanning using the ultrasound probe is performed along the first scanning path and the second scanning path.

12. The image capturing system according to claim 11, wherein:
the ultrasound probe includes:
a transceiver unit that transmits and receives ultrasound waves, and
a gripping part that is continuous with the transceiver unit, and
a width of the gripping part is smaller than a width of an effective image region of the transceiver unit.

13. The image capturing system according to claim 12, wherein:
the ultrasound probe is provided with a depression at a side surface of the gripping part, and
the depression is in contact with the first guide part and the second guide part in a case in which the scanning using the ultrasound probe is performed along the first scanning path and the second scanning path.

14. The image capturing system according to claim 13,
wherein a radius of the depression is larger than a radius of a cross-sectional shape of each of the first guide part and the second guide part.

15. The image capturing system according to claim 11, wherein:
the guide member further includes a third guide part that guides the ultrasound probe along the third scanning path, and
in a case in which a width of the second guide part is denoted by W1, a distance between the second guide part and the third guide part is denoted by W2, a width of the third guide part is denoted by W3, and a width of the effective image regions of the ultrasound probe is denoted by W4,
a relationship of W4>W1/2+W2+W3/2 is satisfied.

16. The image capturing system according to claim 12, further comprising:
an intermediate member that is interposed between the ultrasound probe and an examinee,
wherein the intermediate member is provided at the transceiver unit of the ultrasound probe.

17. A mammography apparatus comprising:
the compression plate according to claim 2.

18. A mammography apparatus comprising:
the compression plate according to claim 3.

19. A mammography apparatus comprising:
the compression plate according to claim 4.

20. A mammography apparatus comprising:
the compression plate according to claim 5.
